(19) United States
(12) Patent Application Publication
(10) Pub. No.: US 2025/0034094 A1
(43) Pub. Date: Jan. 30, 2025

(54) A FAST AND EFFICIENT PROCESS FOR THE PREPARATION OF N-RETINYLIDENE-N-RETINYLETHANOLAMINE (A2E)

(71) Applicants: Purdue Research Foundation, West Lafayette, IN (US); Comell University Center for Technology Licensing at Cornell University (CTL), Ithaca, NY (US)

(72) Inventors: David H. Thompson, West Lafayette, IN (US); Giulia Murbach De Oliveira, West Lafayette, IN (US); Marcelo M. Nociari, New York, NY (US)

(21) Appl. No.: 18/711,740

(22) PCT Filed: Nov. 22, 2022
(86) PCT No.: PCT/US2022/050689

§ 371 Date: May 20, 2024

Related U.S. Application Data
(60) Provisional application No. 63/281,785, filed on Nov. 22, 2021.

(51) Int. Cl. C07D 213/90

(52) U.S. Cl. CPC C07D 213/90

(57) ABSTRACT

A process for the preparation of high quality N-retinylidene-N-retinylethanolamine (A2E) using a continuous flow reaction system is described, wherein the process first provides a mixing point for one equivalent of ATR and about ten equivalents of ethanolamine, followed by later introduction of about twelve equivalents of acetic acid into the stream of the continuous reaction flow at about 25° C. using DMSO as the solvent. The products obtained by this process are also described. A rapid method to access the purity of a sample of A2E is described.

### CROSS-REFERENCE TO RELATED APPLICATIONS

This patent application claims the benefit under 35 U.S.C. § 119(e) of U.S. Provisional Patent Application No. 63/281,785, filed on 22 Nov. 2021, the contents of which are hereby incorporated by reference in their entirety into this disclosure.

### GOVERNMENT SUPPORT

This invention was made with government support under R01EY027422, awarded by the National Institutes of Health. The government has certain rights in the invention.

### TECHNICAL FIELD

The present invention generally relates to a process for the preparation of N-retinylidene-N-retinylethanolamine (A2E). In particular, this fast and efficient process enables an eighty-seven-fold reduction in reaction time, from about 48 hours to about 33 minutes, with an accompanying yield improvement from about 49% to about 78%.

### BACKGROUND

This section introduces aspects that may help facilitate a better understanding of the disclosure. Accordingly, these statements are to be read in this light and are not to be understood as admissions about what is or is not prior art.

The retinal pigment epithelium (RPE) is a cell monolayer that separates the retina from choroid circulation and is vital for the phagocytic recycling of photoreceptor waste, nutrient supply, ionic balance maintenance, and many other critical activities required for proper functioning of the retina¹. Light detection by photoreceptors starts with the conversion of 11-cis-retinal, the prosthetic group sensitive to light in visual pigments, into its isomer, 11-trans-retinal (a.k.a., all-trans-retinal or ATR). ATR then needs to be replaced by another 11-cis-retinal to regenerate rhodopsin as part of the visual cycle process. All-trans or 11-cis retinaldehydes in photoreceptors tend to spontaneously dimerize into lipid bisretinoids that accumulate as retinal lipofuscin. Failures in ABCA4 function, a protein that flips retinaldehydes to the cytosol where deshydrogenases convert them to non-toxic retinols, exacerbate the formation of lipid bisretinoids². As a consequence, lipid bisretinoids end up in the lysosomes of RPE cells, where they remain indefinitely because they are not susceptible to degradation by lysosomal enzymes. The accumulation of bisretinoid-rich lipofuscin in the RPE has been shown to induce cell death³ and is believed to be a key culprit in the etiology of conditions such as Stargardt's disease, cone-rod dystrophy, Best's macular dystrophy and potentially age-related macular degeneration (AMD)⁴.

N-Retinylidene-N-retinylethanolamine (A2E) is the most studied lipid bisretinoid. It forms lipofuscin deposits in the retinal pigment epithelium (RPE), causing vision impairment and blindness in eye conditions, such as Stargardt's disease, cone-rod dystrophy, Best's macular dystrophy and potentially age-related macular degeneration (S. Ben-Shabat et al., 2001). Synthetic A2E is often used for inducing the accumulation of lipofuscins within the lysosomes of RPE cells in culture as an in vitro surrogate of retinal lipofuscin buildup, providing insights into the mechanisms of these eye conditions. Many reports describing the use of synthetic A2E employ material that has been prepared using a one-pot reaction of all-trans-retinal (ATR) and ethanolamine at room temperature for 48 hours (C.A. Parish et al., 1998), and the quality of the product is questionable due to various impurities. There are unmet needs for an efficient, fast, and reliable synthetic process to provide a high-quality material for biomedical researches of eye diseases.

### SUMMARY

This synthesis was revisited by performing a design of experiments (DoE) and high throughput experimentation (HTE) workflow that was tailored to identify the most productive combination of the variables (temperature, solvent, reagent equivalences) for optimization of A2E yield. The DoE findings revealed that the interaction of ethanolamine with acetic acid and ATR was pivotal for the formation of A2E in high yield, likely indicating that imine formation is a critical step in the reaction. Armed with these results, the method was optimized using a microfluidic reactor system before upscaling those conditions for continuous flow synthesis of A2E. This revised method enabled an eighty-seven-fold reduction in reaction time, from 48 hours to 33 minutes, with an accompanying yield improvement from 49% to 78%. Furthermore, a simple method was developed to evaluate the quality of the A2E produced, using optical spectroscopy and LC-MS characteristics to assure that the biological properties observed with A2E samples are not confounded by the presence of occult impurities.

In an embodiment, described herein is a process for the production of N-retinylidene-N-retinylethanolamine (A2E) substantially free of impurities, from ethanolamine and all-trans-retinal (ATR) in the presence of acetic acid, the process comprising the steps of:
a) preparing a first solution consisting essentially of ATR in a first solvent;
b) preparing a second solution consisting essentially of ethanolamine in a second solvent;
c) preparing a third solution consisting essentially of acetic acid in a third solvent;
d) introducing the first, second and third solutions into a continuous flow reactor at a constant flow rate and a constant temperature to yield a fourth solution containing A2E exiting the continuous flow reactor after a residence time in the continuous flow reactor (T_{R}); and
e) purifying the A2E from the fourth solution to yield A2E that is substantially free of impurities.

In another embodiment, described herein is a rapid method to access the purity of a sample of A2E, the method comprising the steps of:
a) dissolving the sample of A2E in alcohol;
b) measuring the ultraviolet absorbances at 439 nm and 339 nm;
c) determining the ratio of the absorbance at 439 nm divided by the absorbance at 339 nm; and
wherein a ratio of about 1.39 indicates the sample of A2E is substantially free of impurities.

The above and other aspects, objects, features, and advantages of the present disclosure will become more apparent and better understood when taken in conjunction with the following description, claims and drawings wherein identical reference numerals have been used, where possible, to designate identical features that are common to the figures.

### BRIEF DESCRIPTION OF THE DRAWINGS

FIG. 1. An outline of the workflow pursued in the Design of Experiments (DoE)-High Throughput Experimentation (HTE) (DoE-HTE) optimization process. An initial 33 DoE was developed to generate a matrix with 27 experiments combining three variables (stoichiometry, solvent and temperature) at three levels to discover the best reaction conditions and the variable interactions. A Biomek i7 liquid handling robot was used to transfer the reagents in proportions required for the DoE into the respective wells of glass vial lined, 96-well plates and sealed.¹¹⁻¹⁵ The plates were then transferred to heating blocks set to 25, 37 and 100° C. and heated for 48 hours. After cooling to room temperature, the solutions were transferred from the three 96-well source plates to one 384-well daughter plate and stored at −80° C. until analysis. The Biomek i7 was then used to pin the solutions from the 384 well plate to a Desorption Electrospray Ionization-Mass Spectrometry (DESI-MS) plate for MS analysis of all the reactions.¹¹⁻¹⁵

FIG. 2. Contour plots derived from analysis of DESI-MS data in 33 DoE-HTE. (A) Interaction of temperature with acetic acid equivalency, where the maximum point occurs at 25° C. and 10 equivalents of acetic acid. (B) Interaction of solvent with acetic acid equivalency, where the maximum point occurs with dimethylsulfoxide (DMSO) and 10 equivalents of acetic acid. (C) Interaction of solvent with temperature, where the maximum point occurs with DMSO and 25° C.

FIG. 3. Pareto chart of the major effects impacting the yield of N-retinylidene-N-retinylethanolamine (A2E). The line represents the cumulative impact of the interactions.

FIG. 4. (A) UV-Vis spectrum of A2E in MeOH after silica gel flash column chromatography purification using step gradient elution with 98:2:0.01 DCM/MeOH/TFA, 90:10:0.01 DCM (CH₂Cl₂)/MeOH/TFA (trifluoroacetic acid) and 2:1:0.01 DCM/MeOH/TFA, in sequence. (B) UV-Vis spectrum of A2E in MeOH after preparative high-performance liquid chromatography (HPLC) purification of the sample in (A) with a ZORBAX ExtendC18 column, 9.4×250 mm, 5 μm, 80 A in a gradient of 85/25 to 95/5 ACN/H₂O for 1 hour with a flow rate of 4 mL/min.

FIG. 5. Continuous flow scale-up configuration. A: all-trans-retinal (ATR), B: Ethanolamine, C: Acetic acid.

FIG. 6. (A) Absorption spectrum obtained by sequential MPLC and preparative HPLC. LC-MS of A2E purified by flash column chromatography. (B) LC-MS was performed using an Eclipse XDB-C18, 4.6×150 mm, 5 μm, 30° C., 95/5 ACN/H₂O with 0.1% TFA isocratically for 10 minutes with a flow rate of 1 mL/min.

FIG. 7. Comparison of three lots of A2E obtained during this study. A2E-#1 (a); A2E-#2 (b); A2E-#3 (c); A2E-#4 (d) is A2E #3 spiked with ATR at a 5:1 A2E: ATR molar ratio; and ATR (e) is shown for reference. The combination of (A) absorbance spectra and (B) fluorescence spectra of the different lots provided a means to readily assess A2E quality. (C) LC-MS data show a dominant 592 Da peak in all the lots analyzed; however, in lots #1 and #2 there are extra peaks that, based on their molecular weights, likely represent heavily and moderately oxidized A2E, respectively. (D) Survival of retinal pigment epithelium cells (ARPE19 cells) after treatment with batches (a), (b), (c), and (d) of A2E and with (e) ATR with no inhibitor (CON), or in the precense of inhibitor Necrostatin 7 (NEC) (or N-acetyl-cysteine (NAC).

FIG. 8. Flow reactor setup in a Labtrix S1 system (Chemtrix BV, Echt, The Netherlands). A: ATR, B: Ethanolamine, C: Acetic acid. The microreactor was a μL Chemtrix 3223 chip with staggered oriental ridge (SOR) mixers. The flow setup was designed so that ATR and ethanolamine would first engage in a T-mixer to initiate the imine formation reaction before it encountered acetic acid in the reactor chip. At the end of the reaction period, each sample was extracted and purified by column chromatography.

FIG. 9. LC-MS was performed using a ZORBAX Extend C-18 column, 2.1×50 mm, 1.8 μm, 30° C., 95/5 CH₃CN/H₂O with 0.1% formic acid isocratically for 20 minutes with a flow rate of 0.3 ml/min with major m/z annotated on respective peaks.

FIG. 10. High resolution mass spectrum detected m/z of 592.4511, corresponding to the exact mass of A2E. ESIMS performed with a fragmentor voltage of 175 V using an Agilent 6550 Q-TOF.

The attached drawings are for purposes of illustration and are not necessarily to scale.

### DETAILED DESCRIPTION

While the concepts of the present disclosure are illustrated and described in detail in the description herein, results in the description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range. In the present disclosure, “substantially free of X” indicates that a compound or material substantially free of X has less than about 5%, less than about 4%, less than about 3%, less than about 2%, less than about 1%, less than about 0.5%, or less than about 0.1% of X in the material. The percentage may be a weight % or a mole % and will be understood based on the context of its use.

In the present disclosure, use of the term “high quality” to describe a compound or material means that the compound or material is substantially free of impurities.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The following clauses disclose several non-limiting embodiments of the disclosure.

1. An efficient synthetic process for the synthesis of high quality N-retinylidene-N-retinylethanolamine (A2E) from acetic acid, ethanolamine, and all-trans-retinal (ATR) through optimization of reaction solvents, molar ratios of the starting materials, reaction temperature, reaction time, and chromatographic purification conditions.

2. The efficient synthetic process for the synthesis of high quality A2E according to clause 1, wherein said high quality of A2E has low content of oxidized species, which adversely impact the biological activities of A2E.

3. The efficient synthetic process for the synthesis of high quality of A2E according to clause 1, wherein said high quality of A2E is measured/characterized according to UV-Vis, NMR and LC-MS data.

4. The efficient synthetic process for the synthesis of high quality of A2E according to clause 3, wherein said high quality of A2E has an UV-Vis spectra having a higher absorbance at about 440 nm than the absorbance at about 331 nm.

5. The efficient synthetic process for the synthesis of high quality of A2E according to clause 1, wherein said high quality of A2E is qualified by the absorption ratio of 439 nm/339 nm, which detects the A2E contamination with oxidized species or ATR. Only highly purified A2E gave similar concentrations from either 339 and 439 OD values. The 439 nm/339 nm absorption ratio of 1.39 was the highest for pure A2E.

6. The efficient synthetic process for the synthesis of high quality of A2E according to clause 1, wherein said process is a continuous flow reaction.

7. The efficient synthetic process for the synthesis of high quality of A2E according to clause 1, wherein said optimized temperature is about 37° C. or lower, preferably at 25° C.

8. The efficient synthetic process for the synthesis of high quality of A2E according to clause 1, wherein said optimized solvent is methanol or dimethyl sulfoxide (DMSO), preferably DMSO.

9. The efficient synthetic process for the synthesis of high quality of A2E according to clause 1, wherein said optimized solvent is not ethanol.

10. The efficient synthetic process for the synthesis of high quality of A2E according to clause 1, wherein said optimized reaction time is achieved by adjusting the flow rate and residence time of the reaction system.

11. The efficient synthetic process for the synthesis of high quality of A2E according to clause 1, wherein said optimized chromatographic purification condition is a sequential medium pressure liquid chromatography (MPLC) and high pressure liquid chromatograpyy (HPLC) purification process.

12. The efficient synthetic process for the synthesis of high quality of A2E according to clause 1, wherein said optimized solvent is methanol or DMSO.

13. The efficient synthetic process for the synthesis of high quality of A2E according to clause 1, wherein said optimized ratios of the starting materials are about 1:10:12 of all-trans-retinal:ethanolamine:acetic acid (ATR:EA:AA).

14. The efficient synthetic process for the synthesis of high quality of A2E according to clause 1, wherein said process provides a fast reaction with a much-improved yield.

15. The efficient synthetic process for the synthesis of high quality of A2E according to clause 1, wherein said process first provides a mixing point for one equivalent of ATR and about ten equivalents of ethanolamine, then followed by introduction of about twelve equivalents of acetic acid down the stream of continuous reaction flow at about 25° C. using DMSO as the solvent.

16. A product manufactured according to the process of clauses 1-15.

The following clauses recite additional non-limiting embodiments of the disclosure.

17. A process for the production of N-retinylidene-N-retinylethanolamine (A2E) substantially free of impurities, from ethanolamine and all-trans-retinal (ATR) in the presence of acetic acid, the process comprising the steps of:
a) preparing a first solution consisting essentially of ATR in a first solvent;
b) preparing a second solution consisting essentially of ethanolamine in a second solvent;
c) preparing a third solution consisting essentially of acetic acid in a third solvent;
d) introducing the first, second and third solutions into a continuous flow reactor at a constant flow rate and a constant temperature to yield a fourth solution containing A2E exiting the continuous flow reactor after a residence time in the continuous flow reactor (TR); and
e) purifying the A2E from the fourth solution to yield A2E that is substantially free of impurities.

17b. The process of clause 17 wherein the first solution and second solution are introduced into a mixing chamber prior to being introduced into the continuous flow reactor with the third solution.

18. The process of any one of the preceding clauses wherein each solvent is independently selected from MeOH or DMSO.

19. The process of any one of the preceding clauses wherein each solvent is MeOH or DMSO.

20. The process of any one of the preceding clauses wherein each solvent is DMSO.

21. The process of any one of the preceding clauses wherein the molar ratio of ATR to ethanolamine is 1 to about 10 and the molar ratio of ATR to acetic acid is 1 to about 12.

22. The process of any one of the preceding clauses wherein the TR is from about 3 minutes to about 120 minutes, such as about 3 minutes to 120 minutes or 3 minutes to about 120 minutes.

23. The process of any one of the preceding clauses wherein the TR is about 33 minutes.

24. The process of any one of the preceding clauses wherein the temperature is from about 25° C. to about 50° C., such as about 25° C. to 50° C. or 25° C. to about 50° C.

25. The process of any one of the preceding clauses wherein the temperature is about 25° C.

26. The process of any one of the preceding clauses wherein step e) comprises sequential application of MPLC and preparative HPLC.

27. The process of any one of the preceding clauses yielding A2E which displays a ratio of the U.V. absorbance at 339 nm to the U.V. absorbance at 439 nm of about 1.39 when dissolved in alcohol.

28. The process of any one of the preceding clauses yielding A2E that is substantially free of oxidized A2E, ATR, the enamine of ATR and ethanolamine, and dihydo-A2E.

29. A rapid method to access the purity of a sample of A2E, the method comprising the steps of:
a) dissolving the sample of A2E in alcohol to yield a solution;
b) measuring the U.V. absorbances of the A2E solution in alcohol at 439 nm and 339 nm;
c) determining the ratio of the absorbance at 439 nm divided by the absorbance at 339 nm; and
d) wherein a ratio of about 1.39 indicates the sample of A2E is substantially free of impurities.

30. The method of clause 29, wherein the A2E is prepared by the process of clause 1 or 2.

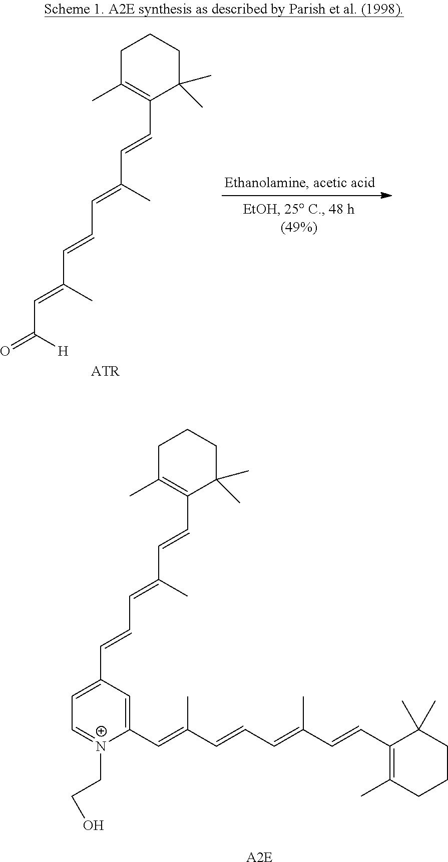

One of the most prevalent and most studied of such lipid bisretinoids is A2E, a pyridinium quaternary amine comprised of two retinaldehyde derived moieties. The most cited article on the synthesis of A2E⁵ uses all-trans-retinal and ethanolamine in a one-step-synthesis (Scheme 1), with one equivalent of acetic acid and ethanolamine in ethanol for 48 hours at room temperature as the best condition for A2E product formation. Scheme 2 shows that the first step in the reaction sequence is the formation of the all-trans-retinal (ATR), ethanolimine, followed by tautomerization to an enamine in a [1,6] proton shift; subsequent addition of a second molecule of ATR produces an iminium ion intermediate. Rearrangement of the iminium ion and subsequent auto-oxidation generates A2E^{4, 5}.

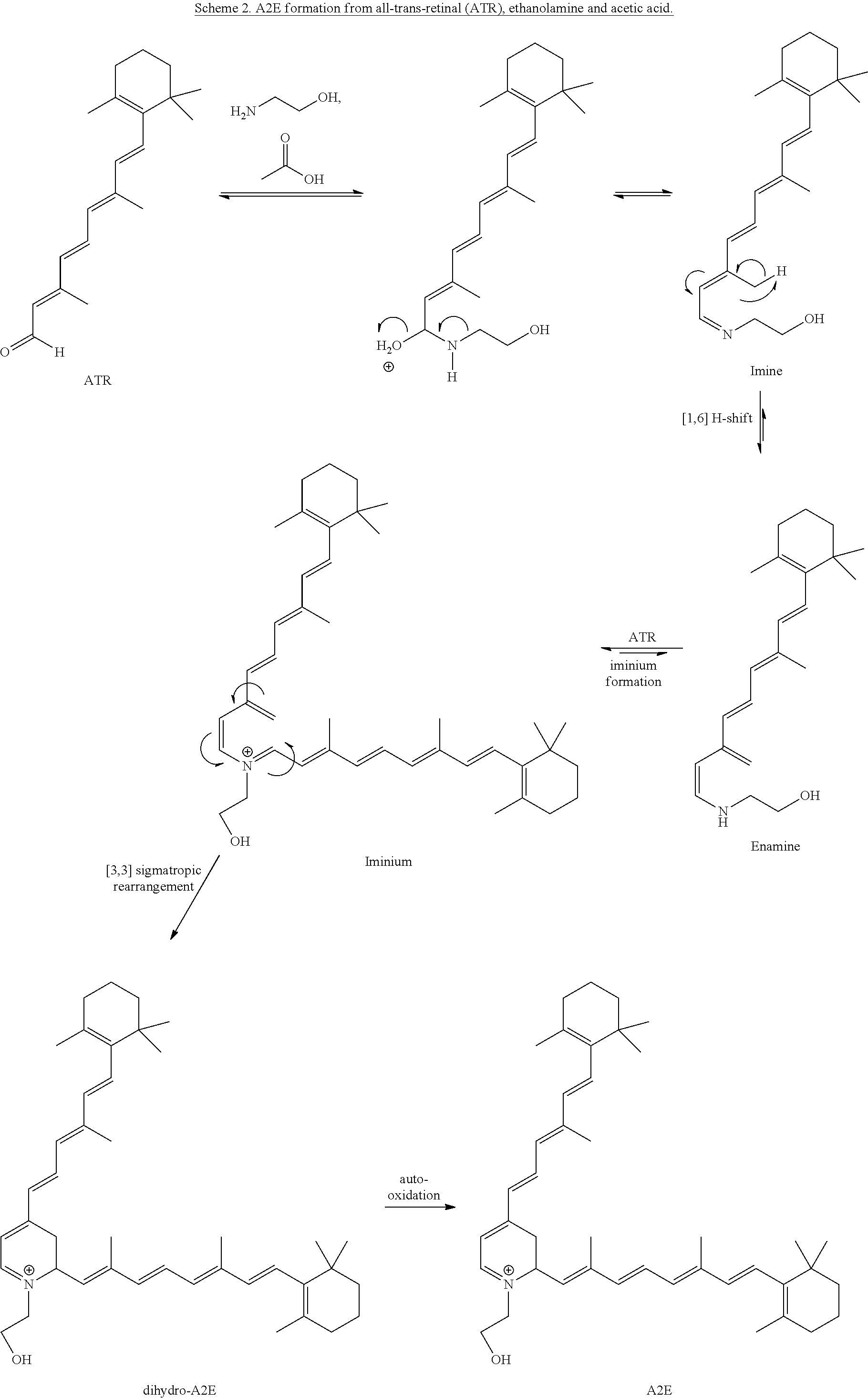

Two aspects of the reaction mechanism are worth noting. In the first step, nucleophilic attack of the aldehyde by ethanolamine will be most favorable under alkaline conditions, since in the presence of acid, the ethanolamine nitrogen will be protonated, thus reducing its nucleophilicity. Conversely, acid conditions favor the second step by protonating the aldehyde oxygen to produce the hydronium leaving group. As the imine formation is an equilibrium, it is necessary to have enough acid to promote the formation of the hydronium ion, but not so much that the nucleophile equilibrium favors full protonation of ethanolamine. Therefore, allowing time for the reaction of all-trans-retinal with ethanolamine prior to the addition of acetic acid and investigation of the equivalence of acetic acid were viewed as crucial parameters to evaluate for optimizing reaction yield.

An additional factor to be considered in the planning of this reaction is the choice of solvent. Jin et al.⁶ studied the effects of different solvents on the extraction of A2E from eyecups. They found that extraction of A2E with different solvents followed by HPLC analysis revealed that A2E is not stable in THF, CHCl₃ or EtOH, but is stable in methanol and DMSO. These findings suggested solvent as another possible parameter to be optimized.

Finally, the role of temperature on reaction efficiency was considered. As the conversion of all-trans-retinal to A2E occurs under physiologic conditions, uncovering whether the A2E yield would increase upon raising the reaction temperature from 22° C. to 37° C. and whether it would be improved at even higher temperatures was investigated.

Given these reaction variables of interest, a design of experiments (DoE) and high throughput experimentation (HTE) strategy was used to improve the synthesis of A2E. DoE is a statistical methodology that aims to identify all major parameters involved in a reaction to reveal how those parameters interact, since reaction parameters are rarely independent of each other⁷⁻⁹. Based on the DoE data obtained, the interplay of reaction parameters can be determined to guide the discovery of optimized conditions.

A key consideration in the DoE approach is the number of experiments to be performed¹⁰. For example, for a 2³ factorial design, with two variables evaluated at three levels, a total of eight unique experiments are required, not including experimental replicates. The number of experiments required grows exponentially with the number of levels applied to the experiment. To simplify the execution of a large number of experiments and reduce costs, high throughput experimentation (HTE) is a valuable technique that is commonly used for data collection. HTE allows for grouping of common operations so that a series of experiments can be rapidly performed in parallel at microscale. This approach also allows for the automation of procedures, such as liquid handling and data analysis, so that hundreds of experiments can be executed simultaneously, and analyzed using quantitative techniques such LC-MS or semi-quantitatively by DESI-MS¹¹⁻¹⁵. HTE also allows for facile and automated replication of experiments, making it the ideal pairing with the DoE approach, since the labor burden for implementing replicates does not increase considerably when the experiments are executed in a microscale format^{16, 17}.

Due to the light and oxidation sensitivity of A2E, the data inputs from the HTE campaign were applied to continuous flow synthesis to provide better control over these parameters. Flow reactions involve the use of automated reagent delivery systems that are continuously mixed within a tubular reactor and collected downstream with control over residence time, flow rate, reactor temperature, light exposure, and reaction O2 content. The use of continuous flow methods can allow for better control over reaction parameters relative to batch syntheses due to the improved surface area-to-volume ratios in flow reactions that enable more efficient mixing and heat transfer. Microfluidic reactors also offer the advantages of safer handling and use of very small quantities of starting materials during the reaction optimization process. For preparative scales, the small-scale setup can be readily upscaled with the same control over mixing and heat transfer¹⁸.

### Results and Discussion

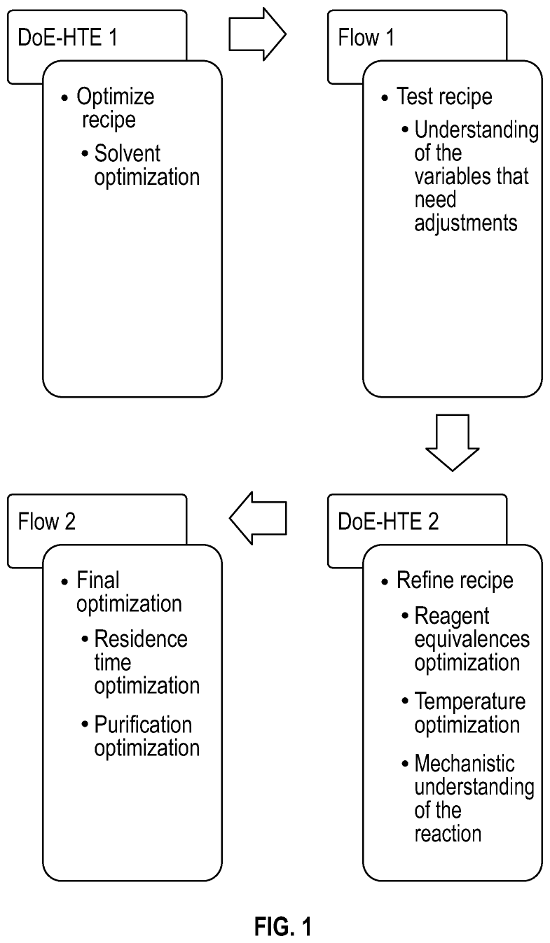

FIG. 1 outlines the workflow pursued in the DoE-HTE optimization process. An initial 3³ DoE was developed to generate a matrix with 27 experiments combining three variables (stoichiometry, solvent and temperature) at three levels to discover the best reaction conditions and the variable interactions. A Biomek i7 liquid handling robot was used to transfer the reagents in proportions required for the DoE into the respective wells of glass vial-lined, 96-well plates and sealed.¹¹⁻¹⁵ The plates were then transferred to heating blocks set to 25, 37 and 100° C. and heated for 48 hours. After cooling to room temperature, the solutions were transferred from the three 96-well source plates to one 384-well daughter plate and stored at −80° C. until analysis. The Biomek i7 was then used to pin the solutions from the 384-well plate to a DESI-MS plate for MS analysis of all the reactions.¹¹⁻¹⁵

The DESI-MS signal intensities for the A2E product peak (592.45 m/z) for each of the 27 reaction conditions were corrected for background signal, and the measured values were scaled from 0-100% with respect to the highest product peak intensity observed (TABLE 1). Evaluation of the HTE findings from this initial 3³ DoE revealed that the best conditions are one equivalent of acetic acid at 37° C. in DMSO.

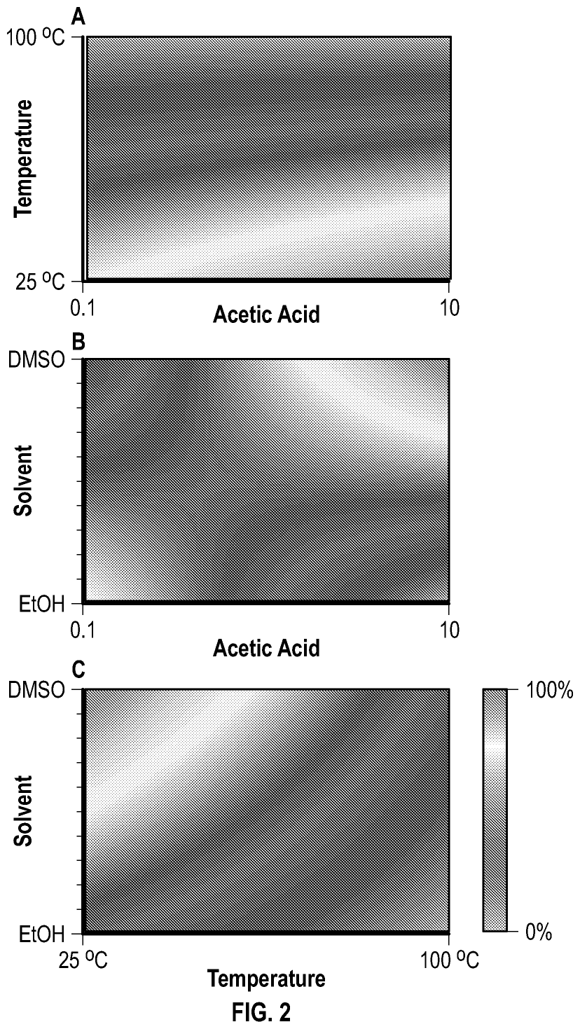

Utilizing Ellistat software, contour plots were generated to predict reaction gradient profiles (FIG. 2). Inputting the ion counts measured in the DESI-MS experiment into the statistical analysis software, a projection of conditions was generated that would give higher ion counts based on the interaction of the factors studied. The contour plots suggest that the preferred conditions for this reaction are DMSO and 10 equivalents of acetic acid at 25° C.

TABLE 1
HTE results, first 33 DoE experiment.

| | Acetic acid | Temp. | | Normalized Ion | |
|---|---|---|---|---|---|
| Run | (eq.) | (° C.) | Solvent | Intensity | % Yield |
| 1 | 0.1 | 25 | EtOH | 120 | 60.7 |
| 2 | 0.1 | 25 | MeOH | 25.2 | 12.7 |
| 3 | 0.1 | 25 | DMSO | 32.1 | 16.1 |
| 4 | 0.1 | 37 | EtOH | 72.4 | 36.4 |
| 5 | 0.1 | 37 | MeOH | 49.5 | 24.9 |
| 6 | 0.1 | 37 | DMSO | 52.8 | 26.5 |
| 7 | 0.1 | 100 | EtOH | 49.4 | 24.8 |
| 8 | 0.1 | 100 | MeOH | 26.4 | 13.3 |
| 9 | 0.1 | 100 | DMSO | 33.3 | 16.7 |
| 10 | 1 | 25 | EtOH | 28.4 | 14.3 |
| 11 | 1 | 25 | MeOH | 41.0 | 20.6 |
| 12 | 1 | 25 | DMSO | 31.2 | 15.7 |
| 13 | 1 | 37 | EtOH | 94.0 | 47.2 |
| 14 | 1 | 37 | MeOH | 77.5 | 38.9 |
| 15 | 1 | 37 | DMSO | 199 | 100 |
| 16 | 1 | 100 | EtOH | 34.9 | 17.5 |
| 17 | 1 | 100 | MeOH | 12.8 | 6.5 |
| 18 | 1 | 100 | DMSO | 28.5 | 14.3 |
| 19 | 10 | 25 | EtOH | 1.30 | 0.7 |
| 20 | 10 | 25 | MeOH | 44.2 | 22.2 |
| 21 | 10 | 25 | DMSO | 114 | 57.2 |
| 22 | 10 | 37 | EtOH | 92.0 | 46.2 |
| 23 | 10 | 37 | MeOH | 23.6 | 11.8 |
| 24 | 10 | 37 | DMSO | 159 | 79.8 |
| 25 | 10 | 100 | EtOH | 12.1 | 6.1 |
| 26 | 10 | 100 | MeOH | 35.3 | 17.7 |
| 27 | 10 | 100 | DMSO | 17.6 | 8.9 |

### Round 1: Continuous Flow Experiments

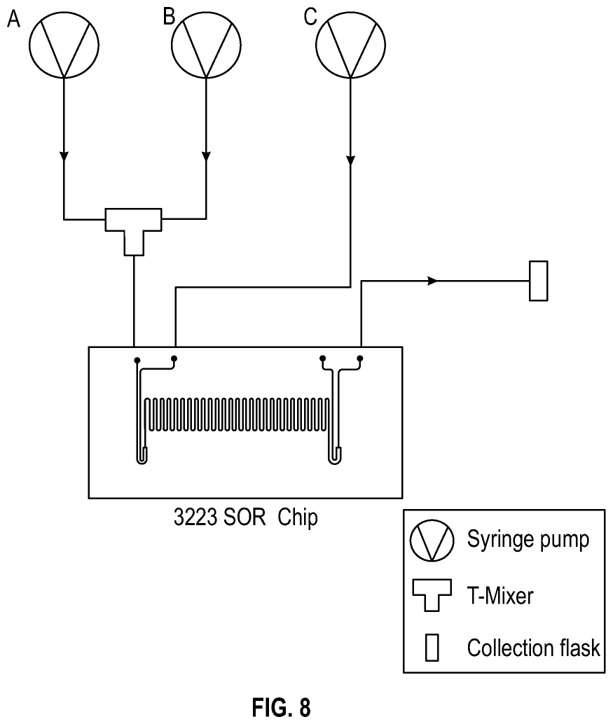

The results obtained from the HTE and DoE analyses were used to guide an initial screen of continuous flow conditions (TABLE 2). The flow setup (FIG. 8) was designed so that ATR and ethanolamine would first engage in a T-mixer to initiate the imine formation reaction before encountering acetic acid in the reactor chip. At the end of the reaction period, each sample was extracted and purified by column chromatography.

One indication of A2E purity is its UV-Vis absorption spectrum. When pure, A2E will have two defined bands: one at about 336 nm and a more intense one around 439 nm^{5, 19, 20}. The intensity of the 439 nm band is important, since ATR, the synthetic precursor and potential contaminate in the isolated A2E fraction, contributes more to the sample absorbance at 330 nm than at 439 nm. Neither reaction generated a very pure A2E sample, even though their NMR spectra showed all the expected peaks for this compound. The difference between the UV-Vis spectra obtained for the two experiments is remarkable, with the experiment run at lower temperature and higher acetic acid equivalency showing clearer bands on 333 nm and 432 nm. This observation suggests that both the decrease in temperature and increase in acetic acid equivalency leads to increased reaction yield.

### Second Round of DoE and HTE

A second DoE was performed with a focus on A2E yield improvement and suppression of byproduct formation. After analyzing the batch experiments that produced A2E in the literature, we observed that there are two different reagent stoichiometries utilized. While Parish et al.⁵ utilizes a 2.27:1:1 ratio of ATR:ethanolamine:acetic acid, Guan et al.²¹ utilizes a 1:19:24 ratio of ATR:ethanolamine:acetic acid.

TABLE 2
HTE results for the 33 DoE equivalences experiment.

| | Equivalents | | | % Yield | % Yield | % Yield |
|---|---|---|---|---|---|---|
| Run | ATR | EA | AA | 25° C. | 37° C. | 50° C. |
| 1 | 2.7 | 1 | 1 | 23.9 | 66.5 | 16.7 |
| 2 | 2.7 | 1 | 12 | 44.2 | 1.8 | 28.6 |
| 3 | 2.7 | 1 | 24 | 22.5 | 54.1 | 13.1 |
| 4 | 2.7 | 10 | 1 | 21.1 | 1.9 | 11.3 |
| 5 | 2.7 | 10 | 12 | 46.9 | 69.1 | 10.8 |
| 6 | 2.7 | 10 | 24 | 26.1 | 3.1 | 4.5 |
| 7 | 2.7 | 19 | 1 | 5.0 | 13.3 | 1.4 |
| 8 | 2.7 | 19 | 12 | 41.1 | 20.4 | 6.1 |
| 9 | 2.7 | 19 | 24 | 32.7 | 22.1 | 7.1 |
| 10 | 1.5 | 1 | 1 | 47.8 | 77.8 | 17.3 |
| 11 | 1.5 | 1 | 12 | 19.8 | 23.3 | 35.1 |
| 12 | 1.5 | 1 | 24 | 14.6 | 16.2 | 8.7 |
| 13 | 1.5 | 10 | 1 | 46.4 | 17.2 | 5.5 |
| 14 | 1.5 | 10 | 12 | 73.8 | 75.6 | 12.9 |
| 15 | 1.5 | 10 | 24 | 13.2 | 20.9 | 2.3 |
| 16 | 1.5 | 19 | 1 | 14.7 | 37.4 | 1.4 |
| 17 | 1.5 | 19 | 12 | 27.9 | 30.8 | 13.2 |
| 18 | 1.5 | 19 | 24 | 65.5 | 9.6 | 4.8 |
| 19 | 1 | 1 | 1 | 80.4 | 18.3 | 32.2 |
| 20 | 1 | 1 | 12 | 21.1 | 16.1 | 33.3 |
| 21 | 1 | 1 | 24 | 13.9 | 42.0 | 7.1 |
| 22 | 1 | 10 | 1 | 71.8 | 52.5 | 20.5 |
| 23 | 1 | 10 | 12 | 100.0 | 44.8 | 15.5 |
| 24 | 1 | 10 | 24 | 23.8 | 16.0 | 3.1 |
| 25 | 1 | 19 | 1 | 48.6 | 13.1 | 0.3 |
| 26 | 1 | 19 | 12 | 19.2 | 33.6 | 9.0 |
| 27 | 1 | 19 | 24 | 22.8 | 18.8 | 18.8 |

ATR = all-trans retinal; EA = ethanolamine; AA = acetic acid.

In order to understand the role of each reagent in the synthesis of A2E, another 33factorial DoE was designed, where the equivalence of each reagent was used as the minimum, intermediate and maximum factors to generate a matrix of 27 unique experiments with DMSO as solvent. We also sought to explore the effect of reaction temperatures on A2E yield, so that each set of 27 replicated experiments was performed at 25, 37 and 50° C. (we lowered the highest temperature in the experiments to 50° C. since the first DoE revealed that 100° C. was detrimental to A2E yield). Comparisons between the A2E product ion intensities produced by all 81experiments indicates that the best reaction condition is one equivalent of ATR, 10 equivalents of ethanolamine and 12 equivalents of acetic acid at 25° C. (TABLE 2).

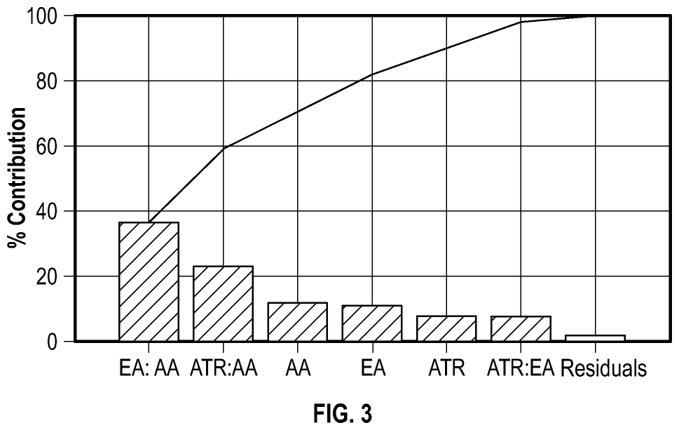

To gain a deeper understanding of the results obtained, a Pareto analysis was generated to detail the importance of each factor and/or combination of factors (FIG. 3). The Pareto results reveal that the two most important factors are the interaction between ethanolamine:acetic acid equivalence and the interaction between ATR:acetic acid equivalence. Based on the mechanistic considerations for imine formation, it was concluded that it was necessary to have a considerable amount of acid to enable leaving group formation, but without reducing the effective concentration of free amine in solution that is available for ATR aldehyde engagement.

### Optimization of Continuous Flow Experiments

Using the results of the second HTE as a guide, flow syntheses at two different flow rates—1 μL/min (i.e., a residence time, TR, of 3.3 minutes) and 0.1 L/min (T_{R}=33 minutes) were performed. TLC analysis of the products showed that increased residence time produced a more intense spot for the product and less intense spots for the by-products, findings that were confirmed by product isolation after flash column chromatography (TABLE 3). The improved yield when the residence time is increased by an order of magnitude is related to the fact that the one-pot synthesis of A2E is a product of five consecutive reactions over 48 hours, such that a T_{R}=3.3 minutes does not provide enough time for all of these reactions to occur. The maximum residence time possible for the experiment was achieved at 33 minutes due to the size limitations of the 3223 reactor and S1 system chosen for the study.

TABLE 3
Continuous flow conditions tested to evaluate residence
time effects on A2E yields (25° C., DMSO).

| | Equivalence | Flow rate (μL/min) | Residence | | A2E |
|---|---|---|---|---|---|
| Exp | ATR:EA:AA | ATR:EA:AA | time (T_{R}) | | Yield |
| 1 | 1:10:12 | 1:1:1 | 3.3 | min | 9% |
| 2 | 1:10:12 | 0.1:0.1:0.1 | 33 | min | 78% |

ATR = all-trans-retinal (0.3 mol/L); EA = ethanolamine (3 mol/L); AA = acetic acid (3.6 mol/L).

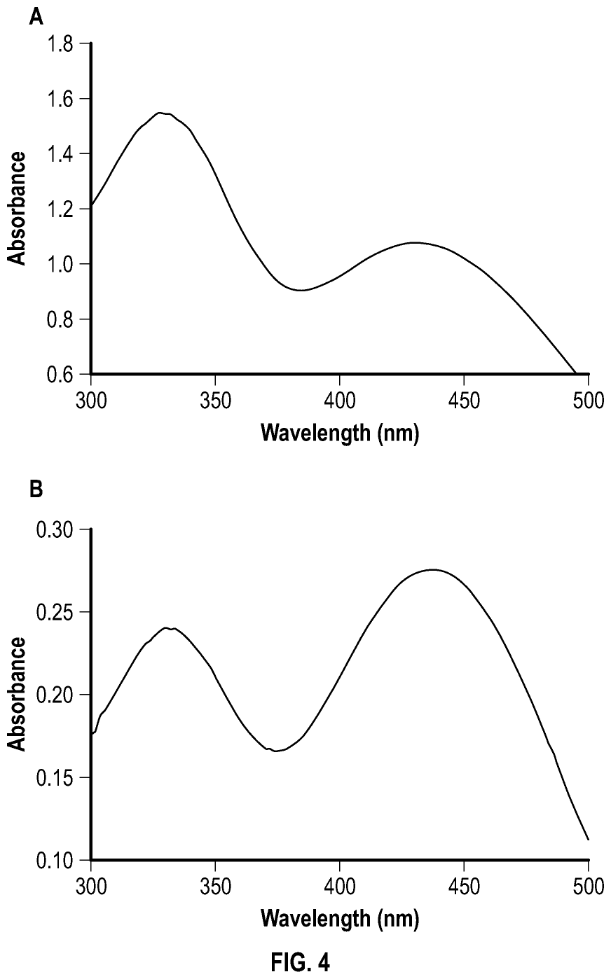

The flash column chromatography purified A2E produced a UV-Vis spectrum (FIG. 4A) with improved absorption bands from that which was obtained in the first round of flow experiments, however, it still did not produce the spectrum desired (a greater intensity for the 439 nm band).

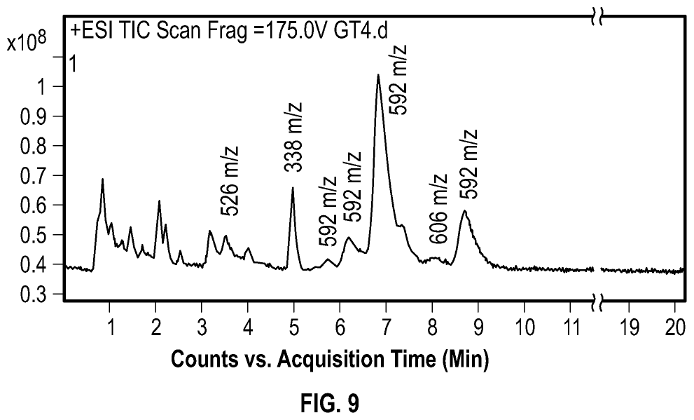

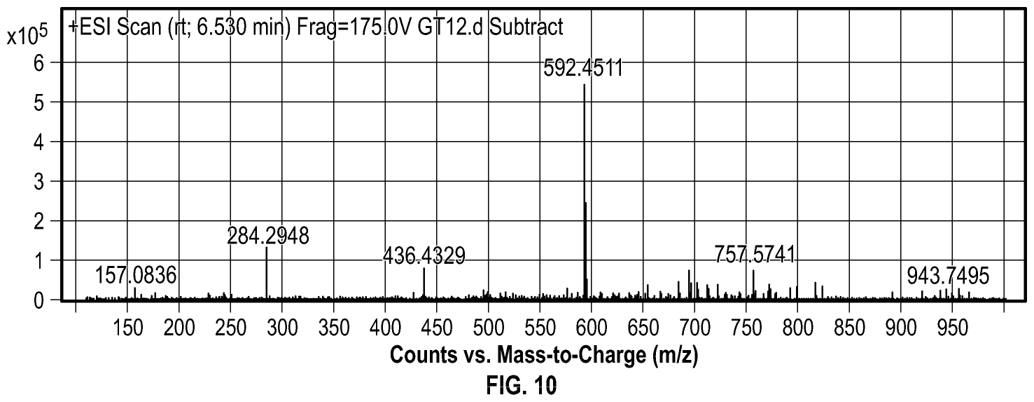

LC-MS analysis (FIG. 9) of the purified A2E isolated by a single flash column chromatography shows that the isolated A2E is still a mixture of over a dozen different compounds. The major peaks shown in the LC-MS data correspond to m/z 592, A2E, another peak with m/z 592 (cis isomer) and m/z 608 (oxidized A2E)²²

Based on these data, the conditions for preparative HPLC were tailored to produce a clear separation between the compounds in the mixture (ZORBAX ExtendC18 column, 9.4×250 mm, 5 μm, 80 A in a gradient of 85/25 to 95/5 Acetonitrile (ACN)/H₂O for 1 hour with a flow rate of 4 mL/min). A2E samples synthesized in flow and purified by this method gave a UV-Vis spectrum with clear peaks at 331 nm and 440 nm (FIG. 4B). Collectively, the UV-Vis, NMR and MS data indicate pure A2E was obtained by this sequential flash and preparative HPLC method.

### Scale-Up of A2E Synthesis in Flow

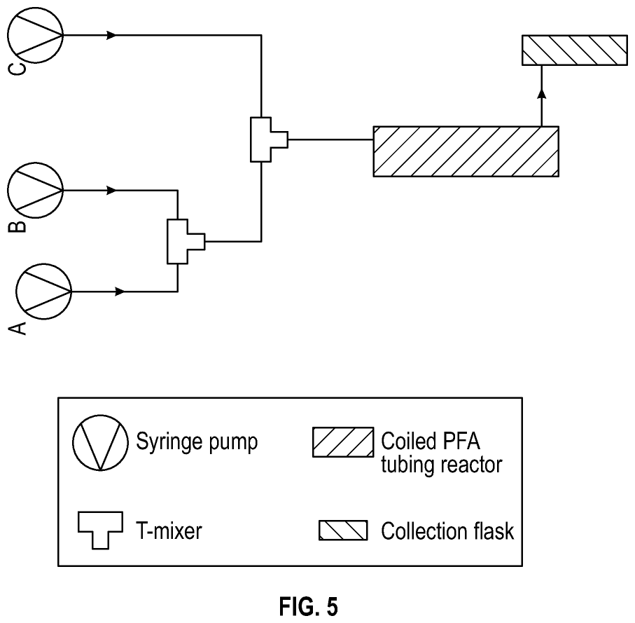

The flow setup used three syringe pumps to deliver the reagents, with ATR and ethanolamine first engaging in a T-mixer; that mixture then encounters acetic acid in a second T-mixer before flowing this final mixture through a coiled PFA tubing reactor (FIG. 5).

Applying the optimized conditions derived from the microscale setup, the reagent equivalencies and residence time were maintained, but three different flow rates of 10, 5 and 2 μL/min were tested. The faster flow rate (10 μL/min) proved to be the most efficient (TABLE 4), a finding that can be attributed to improved mixing with increasing flow rate²³.

TABLE 4
Upscaled conditions for the synthesis of A2E at
25° C. in DMSO at T_{R} = 33 min.

| | Equivalence | Flow rate (μL/min) | A2E |
|---|---|---|---|
| Exp | ATR:EA:AA | ATR:EA:AA | Yield |
| 1 | 1:10:12 | 10:10:10 | 78% |
| 2 | 1:10:12 | 5:5:5 | 50% |
| 3 | 1:10:12 | 2:2:2 | 57% |

ATR = all-trans retinal (0.3 mol/L); EA = ethanolamine (3 mol/L); AA = acetic acid (3.6 mol/L).

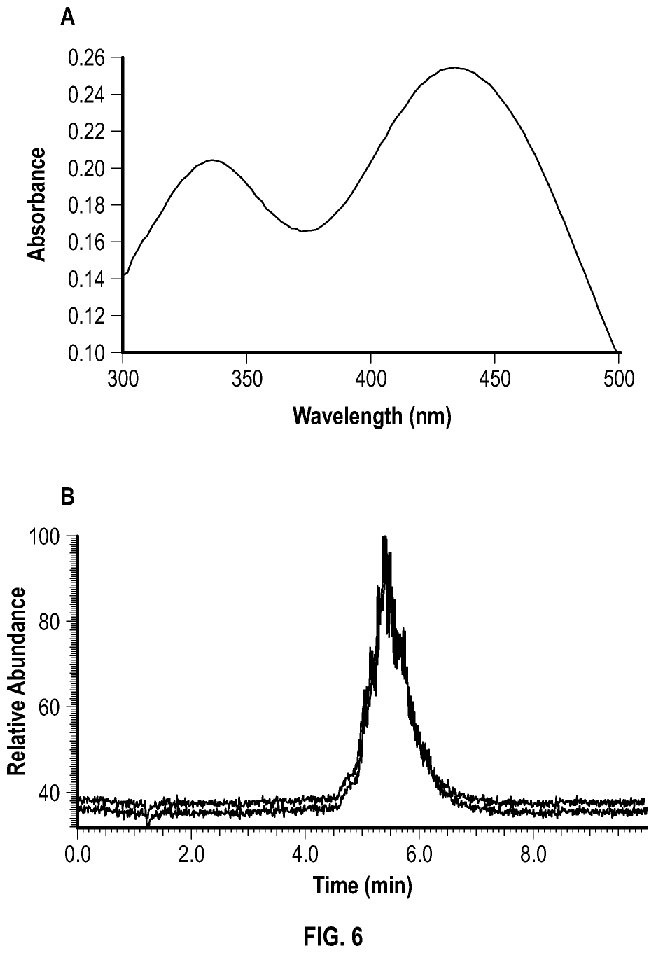

Pure A2E was obtained by collecting the reaction mixture from the flow reactor and isolating the product by sequential medium pressure liquid chromatography (MPLC) and preparative HPLC. The purity of A2E obtained is corroborated by the UV-Vis spectrum obtained (FIG. 6A), NMR (see below) and LC-MS (FIG. 6B). All data matched the results reported in the literature by Sparrow et al.⁵ and Sicre & Cid²⁴, but not Penn et al.²⁵

¹H NMR (CD₃OD, 500 MHz): δ 1.07 and 1.08 (6H each, s, C5-(CH3)₂ and C5′-(CH₃)₂); 1.53 (4H, m, C2-H₂ and C2′-H₂); 1.69 (4H, m, C3-H₂ and C3′-H₂); 1.75 and 1.77 (3H each, s, Cl-CH₃ and Cl′-CH₃); 2.07 3H, s, C9-CH₃); 2.10 (4H, m, C4-H₂ and C4′-H₂); 2.18 (3H, s, C13-CH₃); 2.20 (3H, s, C9′-CH₃); 3.94 (2H, t, CH₂—O); 4.56 (2H, t, N—CH₂); 6.20 (1H, d, C8-H); 6.27 (lH, C10-H); 6.32 (lH, d, C8′-H), 6.37 (lH, d, C7-H); 6.44 (lH, d, C10′-H); 6.57 (lH, d, C7′-H); 6.63 (lH, d, C12-H); 6.72 (lH, s, C14-H); 6.78 (lH, d, C12′-H); 7.15 (lH, dd, C11-H); 7.89 (lH, d, C13′-H); 7.96 (lH, dd, C14′-H); 8.01 (lH, dd, C11′-H); 8.56 (lH, d, C15′-H). Peaks at 4.89 ppm and 3.34 ppm are HOD and MeOD, respectively.

As a further test of the method, two parameters were changed: residence time and solvent. In a first approach, the flow rate, reagent equivalences, temperature, and solvent were maintained, but the residence time was increased four-fold, from 33 minutes to 120 minutes (TABLE 5). The increased residence time only led to the increased production of by-products, with a major UV-Vis peak at 328 nm, a feature that is highly suggestive of increased of A2-DHP-E content^{19, 26}. In the second case, the flow rate, residence time, reagent equivalences and temperature were maintained, but ethanol instead of DMSO was used. This reaction produced a far greater amount of iso-A2E than A2E (TABLE 6).

TABLE 5
Comparison between 33 min and 120 min for residence time, maintaining
flow rate, reagent equivalences, temperature and solvent.

| | | Flowrate | Residence | | |
|---|---|---|---|---|---|
| | Equivalence | (μL/min) | time | | A2E |
| Experiment | ATR:EA:AA | ATR:EA:AA | (T_{R}) | | Yield |
| 1 | 1:10:12 | 10:10:10 | 33 | min | 78% |
| 2 | 1:10:12 | 10:10:10 | 120 | min | 0% |

TABLE 6
Comparison between DMSO and Ethanol, maintaining the flow
rate, residence time, reagent equivalences and temperature

| | | Flow Rate | | | A2E yield/ |
|---|---|---|---|---|---|
| | Equivalence | (μL/min) | Residence | | Iso-A2E |
| Solvent | ATR:EA:AA | ATR:EA:AA | Time | Temp | yield |
| DMSO | 1:10:12 | 5:5:5 | 33 min | 25° C. | 50%/1.3% |
| EtOH | 1:10:12 | 5:5:5 | 33 min | 25° C. | 5%/6.8 |

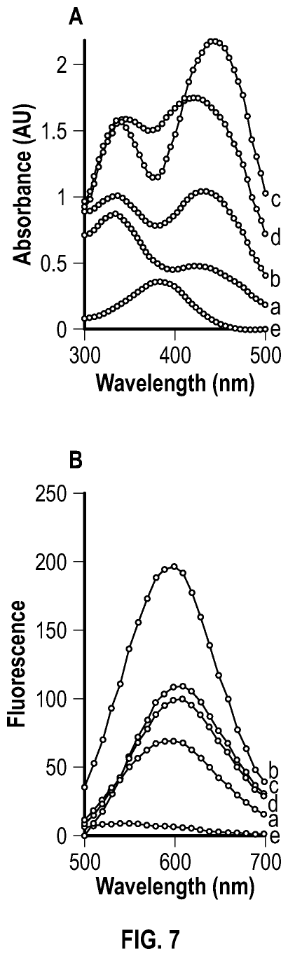

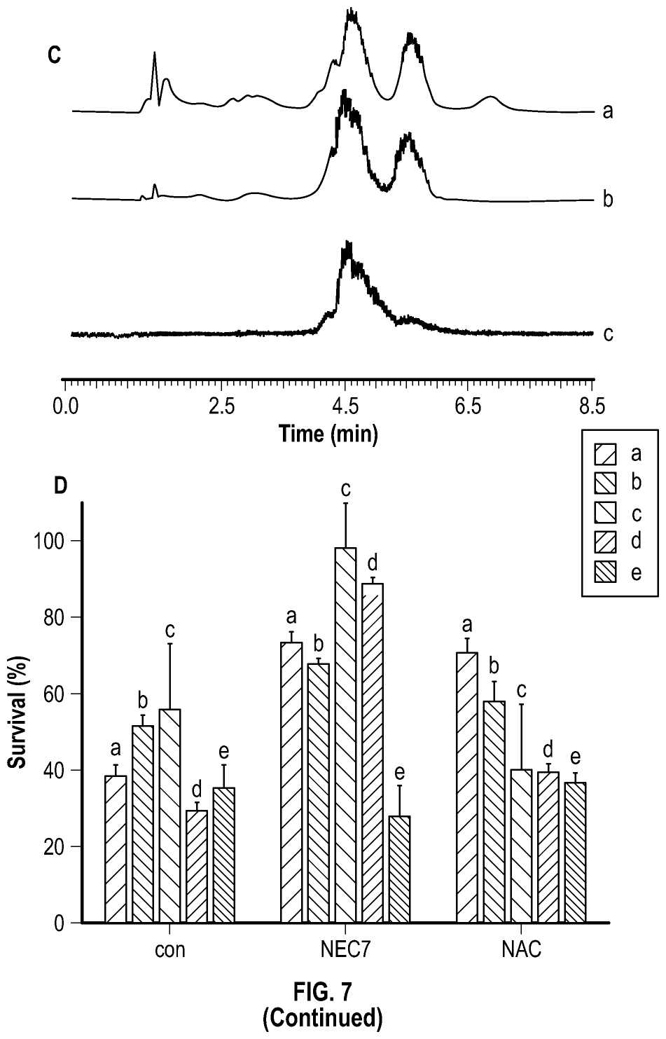

In order to develop an approach to rapidly assess and standardize A2E quality, absorption and fluorescence spectra were used. This method was extremely sensitive and easy for detecting differences in sample quality (FIGS. 7A and 7B). To understand the chemical basis underlying the spectral changes, LC-MS analysis of the different A2E preparations was performed (FIG. 7C and TABLE 7). Two major m/z values were found for these samples: 592, corresponding to A2E, and 608, A2E's oxidized form. The ratio of oxidized and intact A2E's peaks was much larger in the case for Samples #1 (1:8) and #2 (1:3), compared to Sample #3 (1:62) that exhibited a minimal number of impurities. Although all A2E lots were toxic to RPE cells when fed in the dark for 24 hours, the toxicities induced by oxidized lots #1 and #2 were partially protected by antioxidants (NAC), whereas intact A2E toxicity (A2E #3 and A2E #4) was better neutralized with the necroptosis inhibitor, Nec7.²⁷ Thus, the quality of the biological data generated with the different A2E lots was surprisingly affected by the presence of even a small percentage of oxidized material (<5%) and in less degree by residual ATR, leading to confounding biological results (See FIG. 7D). This indicates the importance of the last purification step for the reliability of the biological response induced. TABLE 8 shows the concentrations of A2E inferred from the absorbance readings at 339 nm or 439 nm, using the published molar extinction coefficients.26 It was found that the resulting concentrations from the 339 and 439 nm peaks from the same lot were only coincident when A2E was pure and intact. This observation led to calculation of the 439/339 absorbance-ratio. This ratio was low in oxidized or ATR contaminated samples and increased to a maximum of 1.39 in A2E with the highest integrity and purity. Accordingly, the 439/339 ratio provides a way to easily rank the quality of the A2E preparations.

TABLE 7
Percent Area for all major peaks present in the chromatograms (FIG. 7C) for A2E samples analyzed.

| | | | | | | | | % Area | % Area | Ratio |
|---|---|---|---|---|---|---|---|---|---|---|
| | m/z | m/z | m/z | m/z | m/z | m/z | m/z | oxidized | A2E | oxidized:A2E |
| A2E#1 | 352 | 392 | 608 | 592 | 592 | 592 | 592 | | | |
| % Area | 7.51 | 7.25 | 3.89 | 7.75 | 31.16 | 28.02 | 5.49 | 3.9 | 31.2 | 1:08 |
| A2E#2 | 342 | 472 | 606 | 608 | 592 | 592 | — | | | |
| % Area | 10.76 | 12.94 | 7.08 | 6.56 | 37.98 | 16.19 | — | 13.6 | 38 | 1:03 |
| A2E#3 | 608 | 592 | 592 | — | — | — | — | | | |
| % Area | 1.47 | 91.34 | 7.20 | — | — | — | | 1.47 | 91.34 | 1:62 |
| A2E#4 | 366 | 448 | 608 | 592 | 592 | — | — | | | |
| % Area | 1.39 | 1.22 | 2.83 | 61.75 | 32.81 | — | — | 2.8 | 61.8 | 1:22 |

TABLE 8

| | | |
|---|---|---|
| ABS (AU) | CC (mM) | Ratio |

| A2E# | 339 | 439 | 339 | 439 | 439/339 |
|---|---|---|---|---|---|
| 1 | 0.87 | 0.46 | 17.01 | 6.18 | 0.52 |
| 2 | 1.01 | 1.03 | 19.73 | 13.95 | 1.02 |
| 3 | 1.57 | 2.18 | 30.71 | 29.52 | 1.39 |
| 4 | 1.56 | 1.67 | 30.37 | 22.62 | 1.07 |

The 439 nm/339 nm absorption ratio was the parameter that can most readily detect A2E contamination with oxidized species or ATR. Absorbance data were used to calculate the concentration of the A2E solutions based on published molar extinction coefficients of A2E at 339 and 439 nm²⁴. Only highly purified A2E gave similar concentrations from either 339 or 439 OD value. The 439 nm/339 nm absorption ratio of 1.39 was the highest for pure A2E.

### Conclusions

Based on conflicting literature reports about the synthesis methodology for A2E and the probable mechanism for A2E formation, two 2³ DoE were created, that determined the best reaction conditions for the one-step synthesis of A2E. DMSO, as reported by Jin et al.⁶, reduces the extent of A2E degradation as it is being formed. The ratio of acetic acid to ethanolamine also proved to be a significant factor for improving reaction yield. The reaction also proved to be sensitive to high temperatures, with more efficient reactions occurring at 25° C.

After identifying the preferred reaction conditions, A2E was synthesized on small and large scales using continuous flow reactors. This modification reduced the reaction time from 48 hours to 33 minutes of residence time leading to a greatly improved A2E production. It was also discovered that increased reaction time and EtOH as solvent lead to greater byproduct formation. The optimized condition for large production of A2E was achieved with a flow rate of 10 μL/min and a residence time of 33 minutes, utilizing DMSO as a solvent at 25° C. with an equivalence ratio of 1:10:12 of ATR:EA:AA. These conditions improved the reaction yield from 49%⁵ to 78%.

Finally, different purification methods were investigated in order to obtain A2E in the highest possible purity. The results suggest that a sequential MPLC and HPLC purification process generates highly pure A2E according to UV-Vis, NMR and LC-MS data. It was discovered that the sequential MPLC+HPLC purification sequence is crucial for the correct biological response of the samples, because, surprisingly, the presence of even a small amount of oxidized species appears to result in variable biological performance.

### Experimental Methods

### Reagents.

AlamarBlue® was from Invitrogen. All other reagents were purchased from Sigma-Aldrich and used without further purification.

### NMR Analysis.

NMR spectra were collected using a Bruker AV-III-500-HD NMR spectrometer in CD₃OD and the chemical shifts reported versus TMS.

### DESI-MS Analysis.

High-throughput experiments and Desorption Electrospray Ionization-Mass Spectrometry (DESI-MS) were performed using a previously published method¹⁴. In brief, a

Biomek i7 liquid handling robot was used to prepare the reactions and a LTQ XL (Thermo Scientific) fitted with a DESI 2D stage (Prosolia Inc.) were used to analyze the reaction outcomes. After planning the experiment, the DoE matrix was transferred to a spreadsheet to be inputted on the Xcalibur software (version 3.0) for future use in the DESI-MS. Then, the reagents were transferred into 96-well heating blocks with the desired amounts (in pre-made solutions with the desired solvent). The heating blocks were set to the correct temperatures and, after 48 hours (and cooling of the heating blocks to room temperature), the solutions were transferred to 384-well plates using the i7 robot. The DESI plates were pinned with the i7 robot, and the DESI plate was analyzed with a linear ion trap mass spectrometer fitted with a DESI imaging source. The average ion counts for each combination in the matrix were replicated from three (HTE 2) to eight times (HTE 1) with their respective blanks. The measured ion counts were averaged and normalized against the ion counts measured for the blank regions of the plate. The yields were calculated from the normalized average ion counts and the collected information was inputted into Ellistat software for statistical analysis.

### Small Scale Continuous Flow Synthesis of A2E.

A2E was synthesized using continuous flow methodology in a Labtrix S1 system (Chemtrix BV, Echt, The Netherlands). Three 1 mL stock solutions of 99% DMSO containing ATR (0.3 mol/L, 85 mg), ethanolamine (0.3 mol/L, 18 mg or 3 mol/L, 183 mg) and acetic acid (0.3 mol/L, 18 mg or 3 mol/L, 180 mg or 3.6 mol/L, 216 mg) were prepared, and the solutions were purged with Ar prior to being loaded to three 1 mL Hamilton syringes (Reno, NV), respectively. ATR and ethanolamine were added via a T junction into the same port of a staggered oriented ridge Chemtrix 3223 reactor chip (10 μL), in which acetic acid was also added into a second port. The syringes and chip were connected by FEP tubing (0.8 mm o.d.×0.25 mm i.d., Dolomite Microfluidics). The respective flow rates are reported in TABLES 5 and 6.

The reaction product solutions were extracted with ACN and washed five times with hexane and 1 M NaOAc. The ACN layer was dried under high vacuum. The resulting red solid was purified by silica gel column chromatography using a step elution with 98:2:0.01 CH₂Cl₂ (DCM)/MeOH/TFA, 90:10:0.01 DCM/MeOH/TFA and 2:1:0.01 DCM/MeOH/TFA, in sequence. The product fractions were further purified via semipreparative HPLC with a ZORBAX ExtendC18 column, 9.4×250 mm, 5 μm, 80 A in a gradient of 85/25 to 95/5 ACN/H₂O for 1 hour with a flow rate of 4 mL/min. The product fractions were flash frozen and lyophilized. The resulting yields are reported in TABLES 5 and 6.

### Scale Up Continuous Flow Synthesis of A2E.

Three 5 mL stock solutions of ATR (0.3 mol/L, 426 mg), ethanolamine (3 mol/L, 916 mg) and acetic acid (3.6 mol/L, 1081 mg) were prepared in 99% DMSO, and the solutions purged with Ar prior to loading into three 25 mL Hamilton syringes, respectively. The syringes were mounted onto two Harvard syringe pumps and connected by FEP tubing ( 1/16×0.010 ft, IDEX) to the flow system according to FIG. 5. The flow rates and R_{T} are described in TABLE 4.

After the reactions were complete, the product solutions were extracted with ACN and washed three times with 1 M NaOAc. The ACN layer was dried under high vacuum. The resulting red solid was purified by MPLC normal phase chromatography in gradient mode for 40 minutes, starting with 98:2:0.01 DCM/MeOH/TFA, until 50% MeOH with a flow rate of 15 mL/min. The product fractions were further purified via preparative HPLC with a Waters Prep C18 XBridge column, 30×100 mm, 10 μm, 80 A in a gradient of 85/25 to 95/5 ACN/H₂O for 35 minutes with a flow rate of 40 mL/min. The product fractions were dried under high pressure. The resulting yields are reported in TABLE 4.

### Cell Viability Assays.

ARPE19 cells from ATCC were plated at 80% confluency in 96-well plates and pre-treated for 1 hour with inhibitors (33 μM Necrostatin 7 (Cayman Chemicals): 2 mM N-acetyl-cysteine (NAC) (Sigma): 50 μg/ml phoroglucinol (Sigma)), after which the medium was supplemented with A2E/ATRD or vehicle (control) and cells were incubated in serum-free OptiMEM (Invitrogen) medium for an additional 23 hours at 37° C. To assess viability, 20 μL of 10× AlamarBlue® (Invitrogen) were added per well, and cells were incubated for an additional hour before reading the fluorescence in SpectraMax M5e (Molecular Devices, CA, USA) using 555 nm excitation/585 nm emission.

### UV/VIS Spectral Evaluation.

A2E quality was assessed by diluting the A2E lot into alcohol. Absorbance and fluorescence spectra were determined in 96-well plates with black walls and clear bottoms. Absorbance was measured between 300 and 500 nm and fluorescence between 500 and 700 nm exciting with 410 nm, using a Spectramax M5e.

### LC-MS Analysis.

LC-MS analysis was performed on a Quantum TSQ Discovery mass spectrometer (ThermoScientific) equipped with ThermoScientific autosampler, ThermoScientific mass spectrometry pump and ThermoScientific ESI detector. Fifteen microliters of sample solution were loaded onto the column and eluted isocratically (mobile phase containing 95% acetonitrile, 5% water, and 0.1% TFA). The column used was an Agilent Eclipse XDB-C18 (4.8×150 mm) with a flow rate of 1 mL/min. The mass spectrometer was operated in positive ion mode with spray voltage at 5000 V and capillary at 350° C. The Q1 quadrupole scanned from m/z 50 to 1000.

Those skilled in the art will recognize that numerous modifications can be made to the specific implementations described above. The implementations should not be limited to the particular limitations described. Other implementations may be possible.

It is intended that that the scope of the present methods and compositions be defined by the following claims. However, this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES CITED

1. Kevany, B. M.; Palczewski, K., Phagocytosis of Retinal Rod and Cone Photoreceptors. Physiology 2010, 25 (1), 8-15.
2. Nociari, M. M.; Lehmann, G. L.; Bay, A. E. P.; Radu, R. A.; Jiang, Z. C.; Goicochea, S.; Schreiner, R.; Warren, J. D.; Shan, J. F.; de Beaumais, S. A.; Menand, M.; Sollogoub, M.; Maxfield, F. R.; Rodriguez-Boulan, E., Beta cyclodextrins bind, stabilize, and remove lipofuscin bisretinoids from retinal pigment epithelium. P Natl Acad Sci USA 2014, 111 (14), E1402-E1408.
3. Ben-Shabat, S.; Parish, C. A.; Vollmer, H. R.; Itagaki, Y.; Fishkin, N.; Nakanishi, K.; Sparrow, J. R., Biosynthetic studies of A2E, a major fluorophore of retinal pigment epithelial lipofuscin. J Biol Chem 2002, 277 (9), 7183-7190.
4. Ben-Shabat, S.; Parish, C. A.; Hashimoto, M.; Liu, J. H.; Nakanishi, K.; Sparrow, J. R., Fluorescent pigments of the retinal pigment epithelium and age-related macular degeneration. Bioorg Med Chem Lett 2001, 11 (12), 1533-1540.
5. Parish, C. A.; Hashimoto, M.; Nakanishi, K.; Dillon, J.; Sparrow, J., Isolation and one-step preparation of A2E and iso-A2E, fluorophores from human retinal pigment epithelium. P Natl Acad Sci USA 1998, 95 (25), 14609-14613.
6. Jin, Q. X.; Dong, X. R.; Chen, J. M.; Yao, K.; Wu, Y. L., Effects of organic solvents on two retinal pigment epithelial lipofuscin fluorophores, A2E and all-trans-retinal dimer. J Zhejiang Univ-Sc B 2014, 15 (7), 661-669.
7. Laird, T., Design of experiments (DoE). Org Process Res Dev 2002, 6 (4), 337-337.
8. Lendrem, D.; Owen, M.; Godbert, S., DOE (design of experiments) in development chemistry: Potential obstacles. Org Process Res Dev 2001, 5 (3), 324-327.
9. Smallwood, H. M., Design of Experiments in Industrial Research. Anal Chem 1947, 19 (12), 950-952.
10. Mills, J. E., Design of experiments in pharmaceutical process research and development. Acs Sym Ser 2004, 870, 87-109.
11. Wleklinski, M.; Loren, B. P.; Ferreira, C. R.; Jaman, Z.; Avramova, L.; Sobreira, T. J. P.; Thompson, D. H.; Cooks, R. G., High throughput reaction screening using desorption electrospray ionization mass spectrometry. Chem Sci 2018, 9 (6), 1647-1653.
12. Loren, B. P.; Ewan, H. S.; Avramova, L.; Ferreira, C. R.; Sobreira, T. J. P.; Yammine, K.; Liao, H. Y.; Cooks, R. G.; Thompson, D. H., High Throughput Experimentation Using DESI-MS to Guide Continuous-Flow Synthesis. Sci Rep-Uk 2019, 9.
13. Ewan, H. S.; Biyani, S. A.; DiDomenico, J.; Logsdon, D.; Sobreira, T. J. P.; Avramova, L.; Cooks, R. G.; Thompson, D. H., Aldol Reactions of Biorenewable Triacetic Acid Lactone Precursor Evaluated Using Desorption Electrospray Ionization Mass Spectrometry High-Throughput Experimentation and Validated by Continuous Flow Synthesis. Acs Comb Sci 2020, 22 (12), 796-803.
14. Biyani, S. A.; Qi, Q. Q.; Wu, J. Z.; Moriuchi, Y.; Larocque, E. A.; Sintim, H. O.; Thompson, D. H., Use of High-Throughput Tools for Telescoped Continuous Flow Synthesis of an Alkynylnaphthyridine Anticancer Agent, HSN608. Org Process Res Dev 2020, 24 (10), 2240-2251.
15. Biyani, S. A. M., Y.W.; Thompson, D.H, Advancement in Organic Synthesis Through High Throughput Experimentation. Chemistry—Methods 2021, 1, 323-339.
16. Shevlin, M., Practical High-Throughput Experimentation for Chemists. Acs Med Chem Lett 2017, 8 (6), 601-607.
17. Mennen, S. M.; Alhambra, C.; Allen, C. L.; Barberis, M.; Berritt, S.; Brandt, T. A.; Campbell, A. D.; Castanon, J.; Cherney, A. H.; Christensen, M.; Damon, D. B.; de Diego, J. E.; Garcia-Cerrada, S.; Garcia-Losada, P.; Haro, R.; Janey, J.; Leitch, D. C.; Li, L.; Liu, F. F.; Lobben, P. C.; MacMillan, D. W. C.; Magano, J.; McInturff, E.; Monfette, S.; Post, R. J.; Schultz, D.; Sitter, B. J.; Stevens, J. M.; Strambeanu, J. I.; Twilton, J.; Wang, K.; Zajac, M. A., The Evolution of High-Throughput Experimentation in Pharmaceutical Development and Perspectives on the Future. Org Process Res Dev 2019, 23 (6), 1213-1242.
18. Plutschack, M. B.; Pieber, B.; Gilmore, K.; Seeberger, P. H., The Hitchhiker's Guide to Flow Chemistry (II). Chem Rev 2017, 117 (18), 11796-11893.
19. Wu, Y. L.; Li, J.; Yao, K., Structures and biogenetic analysis of lipofuscin bis-retinoids. J Zhejiang Univ-Sc B 2013, 14 (9), 763-773.
20. Park, S. I.; Park, S. C.; Kim, S. R.; Jang, Y. P., Two-step Purification Method for Aging Pigments A2E and Iso-A2E Using Medium Pressure Liquid Chromatography. B Korean Chem Soc 2016, 37 (9), 1541-1544.
21. Guan, Z. Q.; Li, Y. W.; Jiao, S. L.; Yeasmin, N.; Rosenfeld, P. J.; Dubovy, S. R.; Lam, B. L.; Wen, R., A2E Distribution in RPE Granules in Human Eyes. Molecules 2020, 25, 1413.
22. Gutierrez, D. B.; Blakeley, L.; Goletz, P. W.; Schey, K. L.; Hanneken, A.; Koutalos, Y.; Crouch, R. K.; Ablonczy, Z., Mass spectrometry provides accurate and sensitive quantitation of A2E. Photochem Photobio Sci 2010, 9 (11), 1513-1519.
23. Samaddar, S.; Mazur, J.; Boehm, D.; Thompson, D. H., Development And In Vitro Characterization Of Bladder Tumor Cell Targeted Lipid-Coated Polyplex For Dual Delivery Of Plasmids And Small Molecules. Int J Nanomed 2019, 14, 9547-9561.
24. Sicre, C.; Cid, M. M., Convergent stereoselective synthesis of the visual pigment A2E. Org Lett 2005, 7 (25), 5737-5739.
25. Penn, J.; Mihai, D. M.; Washington, I., Morphological and physiological retinal degeneration induced by intravenous delivery of vitamin A dimers in rabbits. Dis Model Mech 2015, 8 (2), 131-8.
26. Sparrow, J. R.; Kim, S. R.; Wu, Y. L., Experimental Approaches to the Study of A2E, a Bisretinoid Lipofuscin Chromophore of Retinal Pigment Epithelium. Methods Mol Biol 2010, 652, 315-327.
27. Pan, C., Banerjee, K., Lehmann, Almeida, D., Hajjar, K.A., Benedicto, I., Jiang, Z., Radu, R.A., Thompson, D.H., Rodriguez-Boulan, E., Nociari, M.M., unpublished data.

1. A process for the production of N-retinylidene-N-retinylethanolamine (A2E), which is substantially free of impurities, from ethanolamine and all-trans-retinal (ATR) in the presence of acetic acid, the process comprising the steps of:
a) preparing a first solution consisting essentially of ATR in a first solvent;
b) preparing a second solution consisting essentially of ethanolamine in a second solvent;
c) preparing a third solution consisting essentially of acetic acid in a third solvent;
d) introducing the first, second and third solutions into a continuous flow reactor at a constant flow rate and a constant temperature to yield a fourth solution containing A2E exiting the continuous flow reactor after a residence time in the continuous flow reactor (T_{R}); and
e) purifying the A2E from the fourth solution to yield A2E that is substantially free of impurities.

2. The process of claim 1, wherein the first solution and the second solution are introduced into a mixing chamber prior to being introduced into the continuous flow reactor with the third solution.

3. The process of claim 1, wherein each solvent is independently selected from methanol (MeOH) and dimethyl sulfoxide (DMSO).

4. The process of claim 3, wherein each solvent is MeOH.

5. The process of claim 3, wherein each solvent is DMSO.

6. The process of claim 1, wherein the molar ratio of ATR to ethanolamine is 1 to about 10 and the molar ratio of ATR to acetic acid is 1 to about 12.

7. The process of claim 1, wherein the T_{R} is from about 3 minutes to about 120 minutes.

8. The process of claim 7, wherein the T_{R} is about 33 minutes.

9. The process of claim 1, wherein the temperature is from about 25° C. to about 50° C.

10. The process of claim 9, wherein the temperature is about 25° C.

11. The process of claim 1, wherein step e) comprises sequential application of medium pressure liquid chromatography (MPLC) and preparative high pressure liquid chromatography (HPLC).

12. The process of claim 11, yielding A2E, which displays a ratio of ultraviolet absorbance at 339 nm to ultraviolet absorbance at 439 nm of about 1.39 when dissolved in alcohol.

13. The process of claim 11, yielding A2E that is substantially free of oxidized A2E, ATR, the enamine of ATR and ethanolamine, and dihydro-A2E.

14. A rapid method to access the purity of a sample of A2E, the method comprising the steps of:
a) dissolving the sample of A2E in alcohol to yield a solution;
b) measuring ultraviolet absorbances of the A2E solution in alcohol at 439 nm and 339 nm;
c) determining the ratio of the absorbance at 439 nm divided by the absorbance at 339 nm; and
wherein a ratio of about 1.39 indicates the sample of A2E is substantially free of impurities

15. The method of claim 14, wherein the A2E is prepared by the process of claim 1.

16. The process of claim 2, wherein each solvent is independently selected from methanol (MeOH) and dimethyl sulfoxide (DMSO).

17. The process of claim 16, wherein each solvent is MeOH.

18. The process of claim 16, wherein each solvent is DMSO.

19. The process of claim 2, wherein the molar ratio of ATR to ethanolamine is 1 to about 10 and the molar ratio of ATR to acetic acid is 1 to about 12.

20. The process of claim 2, wherein the T_{R} is from about 3 minutes to about 120 minutes.

21. The process of claim 20, wherein the T_{R} is about 33 minutes.

22. The process of claim 2, wherein the temperature is from about 25° C. to about 50° C.

23. The process of claim 22, wherein the temperature is about 25° C.

24. The process of claim 2, wherein step e) comprises sequential application of medium pressure liquid chromatography (MPLC) and preparative high pressure liquid chromatography (HPLC).

25. The process of claim 24, yielding A2E, which displays a ratio of ultraviolet absorbance at 339 nm to ultraviolet absorbance at 439 nm of about 1.39 when dissolved in alcohol.

25. The process of claim 24, yielding A2E that is substantially free of oxidized A2E, ATR, the enamine of ATR and ethanolamine, and dihydro-A2E.

26. The method of claim 14, wherein the A2E is prepared by the process of claim 2.
